(19) United States
(12) Patent Application Publication
(10) Pub. No.: US 2011/0319810 A1
(43) Pub. Date: Dec. 29, 2011

(54) Curved Infusion Sleeve

(75) Inventor: Ziad R. Ghannoum, Trabuco Canyon, CA (US)

(73) Assignee: ALCON RESEARCH, LTD., Fort Worth, TX (US)

(21) Appl. No.: 12/821,375

(22) Filed: Jun. 23, 2010

(51) Int. Cl. A61F 9/007; B23P 17/04

(52) U.S. Cl. 604/22; 29/428

(57) ABSTRACT

An infusion sleeve, infusion sleeve assembly, and method of making the same are disclosed. An exemplary infusion sleeve assembly may include a tubular body and a tip inserted into the body. The tubular body may include an insertion end and an attachment end. The insertion end may define a tip aperture and a first fluid aperture adjacent the tip aperture, while the attachment end may be configured to be secured to a surgical tool for fluid exchange therewith. The tubular body includes a curved portion between the insertion end and attachment end such that a first axis of the insertion end is angled relative to a second axis of the attachment end. Further, the tubular body may define a substantially smooth surface throughout the curved portion.

### BACKGROUND

Some types of cataract surgery employ a machine with a handpiece equipped with a tip that vibrates at ultrasonic frequencies to emulsify lens material. A dual irrigation-aspiration (I-A) probe may be subsequently employed to aspirate or remove peripheral cortical material using an irrigation solution. In known irrigation systems, irrigation solution is supplied using an infusion sleeve that is inserted into an incision. Apertures in the sleeve provide for simultaneously supplying irrigation fluid to the surgical site and removal/aspiration of cortical material.

Intraocular infusion pressure must generally be carefully controlled during the surgery. Irrigation solution is commonly used to maintain both the anatomic and physiologic integrity of intraocular tissues during surgery by maintaining an equal volumetric flow of the irrigation and aspiration flows.

Infusion sleeves are typically formed of a flexible material to allow fitting the sleeve to a variety of surgical tools having varied shapes and configurations. Further manufacturing efficiencies dictate that known infusion sleeves be formed in the simplest configuration possible, i.e., a straight shape that may be flexed into a curved position. While known infusion sleeves are therefore capable of fitting a wide variety of surgical tools, this may also cause distortion of incisions where there is a difference between the initial shape of the infusion sleeve and the tool. More specifically, where an infusion sleeve is deflected out of its natural position after installation to a surgical tool, the surface of the sleeve may be bunched, stretched, or otherwise distorted. Distortions of the sleeve may risk distortion of an incision or wound operated on with the surgical tool, even where volumetric flow of the aspiration and irrigation flows are carefully monitored and equalized.

Accordingly, there is a need for an improved infusion sleeve that allows for reduced distortion of an incision or wound during insertion and/or withdrawal of the infusion sleeve to/from the incision.

### BRIEF DESCRIPTION OF THE DRAWINGS

While the claims are not limited to the illustrated examples, an appreciation of various aspects is best gained through a discussion of various examples thereof. Referring now to the drawings, illustrative examples are shown in detail. Although the drawings represent the various examples, the drawings are not necessarily to scale and certain features may be exaggerated to better illustrate and explain an innovative aspect of an example. Further, the examples described herein are not intended to be exhaustive or otherwise limiting or restricting to the precise form and configuration shown in the drawings and disclosed in the following detailed description. Exemplary illustrations of the present invention are described in detail by referring to the drawings as follows.

FIG. 1 illustrates a perspective view of an exemplary infusion sleeve assembly;

FIG. 2 illustrates a close-up side view of the exemplary infusion sleeve assembly of FIG. 1;

FIG. 3A illustrates a partial section view of the exemplary infusion sleeve assembly of FIG. 1;

FIG. 3B illustrates a close-up of the partial section view of FIG. 3A: and

FIG. 4 illustrates a process flow diagram of an exemplary method of making an infusion sleeve assembly.

### DETAILED DESCRIPTION

Various exemplary illustrations of an infusion sleeve, infusion sleeve assembly, and method of making the same are disclosed. An exemplary infusion sleeve assembly may include a tubular body and a tip inserted into the body. The tubular body may include an insertion end and an attachment end. The insertion end may define a tip aperture and a first fluid aperture adjacent the tip aperture, while the attachment end may be configured to be secured to a surgical tool for fluid exchange therewith. The tubular body includes a curved portion between the insertion end and attachment end such that a first axis of the insertion end is angled relative to a second axis of the attachment end. Further, the tubular body may define a substantially smooth surface throughout the curved portion.

An exemplary method may generally include forming a tubular body of a single material, including establishing an insertion end of the body with a tip aperture and a first fluid aperture adjacent the tip aperture. The method may further include establishing an attachment end of the body opposite the insertion end, where the attachment end is configured to be secured to a surgical tool for fluid exchange therewith. The exemplary method further includes providing a curved portion between the insertion end and attachment end such that a first axis of the insertion end is angled relative to a second axis of the attachment end. The single material may define a substantially smooth body surface throughout the curved portion.

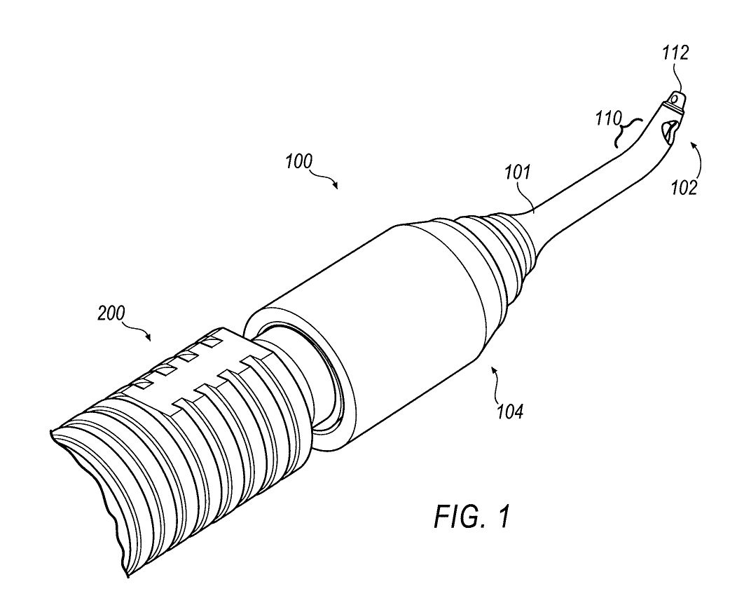

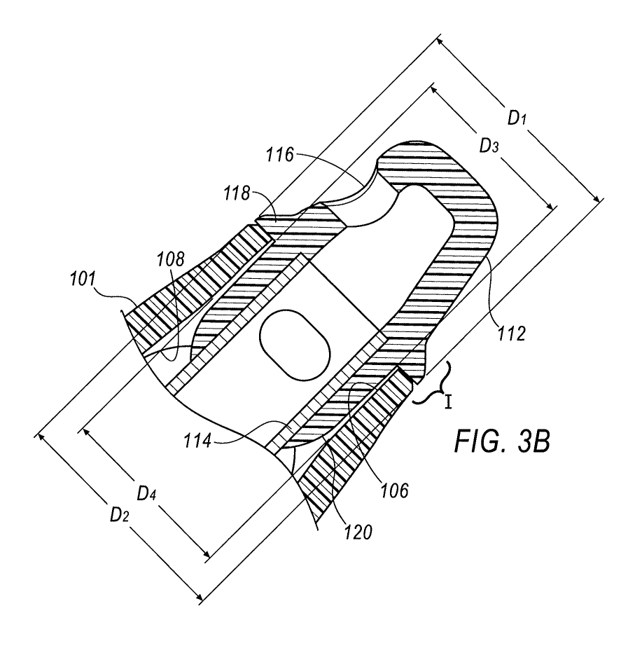

Turning now to FIGS. 1, 2, 3A, and 3B, an exemplary infusion sleeve assembly 100 is shown. The exemplary infusion sleeve 100 generally includes a tubular body 101 and a tip 112 that may be inserted into the tubular body 101. The tubular body 101 may generally be formed of a single material and is defined by an insertion end 102 and an attachment end 104 opposite the insertion end 102. As best seen in FIG. 3A, the insertion end 102 defines a tip aperture 106 and a first fluid aperture 108 adjacent tip aperture 106. The attachment end 104 is generally configured to be secured to a surgical tool 200 for fluid exchange with the surgical tool 200. For example, the infusion sleeve assembly 100 may be generally configured to provide irrigation and aspiration to an incision or wound formed in an ocular cavity, e.g., during cataract surgery, as will be described further below.

The tubular body 101 includes a curved portion 110 between the insertion end 102 and the attachment end 104. Accordingly, the insertion end 102 and attachment end 104 are generally angled with respect to one another. More specifically, as best seen in FIG. 3A, a first axis A-A of the insertion end forms a predetermined angle α relative to a second axis B-B of the attachment end 104. The angled infusion sleeve assembly 100 generally allows greater user comfort during cataract surgery than a straight infusion sleeve assembly. More specifically, a user typically must hold the tool and/or infusion sleeve in a position normal to an ocular incision, e.g., vertically where the patient is lying horizontally during a surgical procedure. As the user often stands to the side of the patient and/or operating site, an angled construction permits the user greater ease of use of the tool because the angled construction allows the tool to extend at least partially in a lateral direction that provides an easier grip for the user. Furthermore, the tubular body 101 may be formed generally of a single material, and may further define a substantially smooth body surface S throughout the curved portion 110.

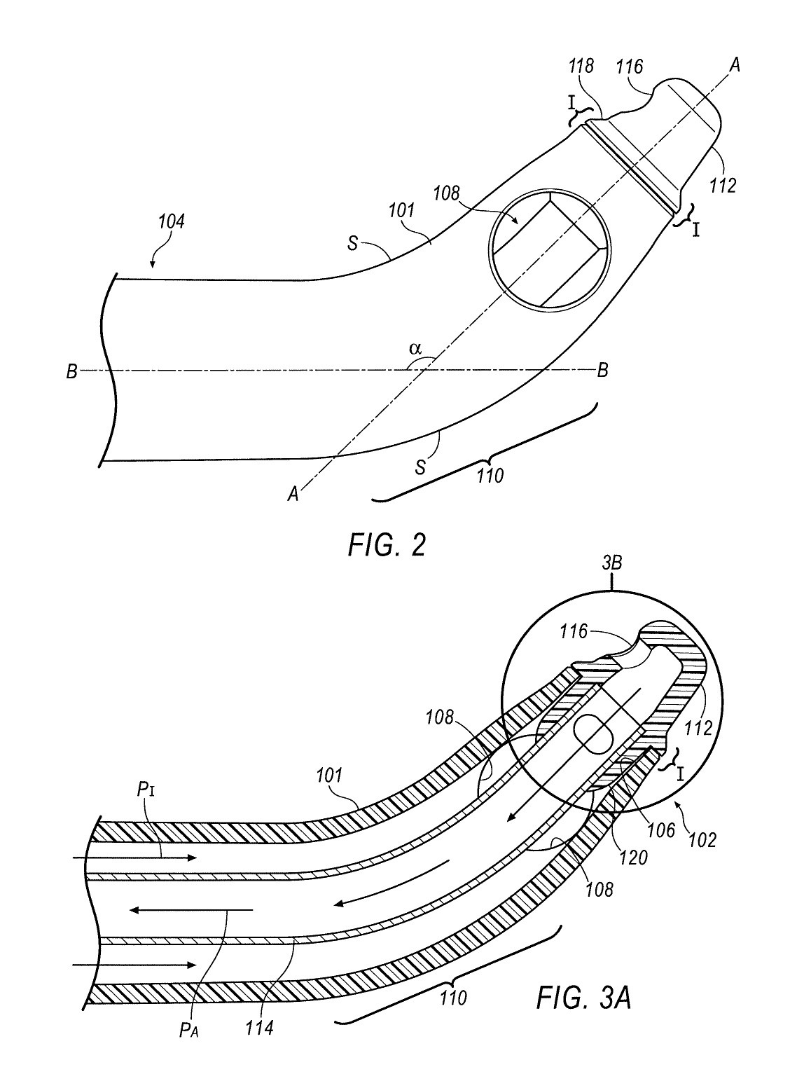

The infusion sleeve assembly 100 also includes a tip 112 that, as best seen in FIGS. 2, 3A, and 3B, may be inserted into the tip aperture 106 of the tubular body 101. The tip 112 generally receives an aspiration tube 114 that may extend from the surgical tool 200 within the tubular body 101. The tip 112 also defines a second fluid aperture 116. The second fluid aperture 116 may be radially spaced away from the first fluid aperture 108 of the tubular body 101 about the axis A-A, in order to minimize interference between fluid flowing out of the first fluid aperture 108 and fluid flowing into the second aperture 116. For example, as best seen in FIG. 2, the first fluid aperture 108 is spaced radially about the axis A-A from the second aperture 116 approximately 90 degrees. The tip 112 is thus generally configured to aspirate fluid from a surgical site by drawing in fluid via fluid aperture 116 and allowing it to be drawn away from the site through the aspiration tube 114.

As best seen in FIG. 3B, the tip 112 defines a generally flared shoulder portion 118 that is positioned adjacent the insertion end 102 when tip 112 is engaged with infusion sleeve assembly 100. The flared shoulder portion 118 generally provides for a smooth interface between the tubular body 101 and the tip 112. In some exemplary illustrations, the flared shoulder portion 118 defines an outer diameter D₁ that is substantially equal to an outer diameter D₂ of the insertion end of 102. Furthermore, any longitudinal gap between the flared shoulder portion 118 and the insertion end 102 may also be minimized or substantially eliminated. Accordingly, an interface between the tip 112 and the tubular body 101 is substantially smooth, thereby minimizing any distortion of an incision into which the infusion sleeve assembly 100 is inserted.

In one exemplary illustration, the tip 112 may include a male connector 120 that is inserted into the tip aperture 106 of the tubular body 101. The male connector 120 may also define an outer diameter D₃ that is substantially equal to an inner diameter D₄ of the insertion end 102 of the tubular body 101 to allow insertion and retention of the tip 112 into the tip aperture 106. Other suitable connection mechanisms may be employed. The interface between the tubular body 101, the tip 112, and the aspiration tube 114 may generally define a fluid-tight interface that allows irrigation fluid to be supplied to a surgical site via an irrigation path P_{I} that flows to the first fluid aperture 108 of the tubular body 101, as best seen in FIG. 3A. Additionally, fluid may be generally simultaneously drawn away from the surgical site via an aspiration path P_{A} generally defined by second fluid aperture 116 and the aspiration tube 114. In some exemplary procedures, a volume of fluid supplied via the irrigation path P_{I} is substantially equal to a volume of fluid drawn away via the aspiration path P_{A}, thereby minimizing or preventing entirely any volume distortion of an ocular cavity associated with the surgical site. The tip 112 may be formed of any material that is convenient. In one exemplary illustration, the tip 112 is formed of an injection molded nylon or plastic material.

As described above, the tubular body 101 may be formed of a single material. In some exemplary illustrations, the tubular body 101 is formed of a flexible and/or resilient material that allows for substantial elastic bending and/or stretching of the material without permanently changing the shape of the tubular body 101, e.g., silicone. The flexible and/or resilient property of the material may allow fitting the tubular body 101 onto the surgical tool 200 and/or aspiration tube 114.

The tubular body 101 includes a curved portion 110 between the insertion end 102 and an attachment end 104 that is generally curved such that the insertion end 102 is angled with respect to the attachment end 104. As best seen in FIGS. 2 and 3A, the curved portion 110 may generally define a smooth surface S on the outer portion of the tubular body 101 throughout the curved portion 110. In other words, there are no surface undulations or discontinuities in the surface S of the tubular body 101, such as may occur when a resilient or flexible material is deflected out of a natural shape, thereby minimizing or preventing entirely any distortion of an incision during insertion or withdrawal of the infusion sleeve assembly 100.

In one exemplary illustration, the smooth outer surface S is created by forming the tubular body in a curved position during the forming process. In other words, the tubular body 101 may be formed such that a natural, e.g., undeflected, position of the tubular body 101 is a curved position such as that shown in the Figures. In another exemplary illustration, the material from which the tubular body is formed may define a substantially constant strain in the single material throughout a curved portion 110 thereof. More specifically, a tension and/or compression of the material as mounted on a curved aspiration tube, e.g., aspiration tube 114, may be substantially equal throughout the curved portion 110. Accordingly, the tubular body 101 exhibits little, if any, surface discontinuities, distortions, or undulations in the surface S that might otherwise cause distortion of an incision into which the infusion sleeve assembly 100 is inserted. By comparison, a flexible and/or resilient material that exhibits bunching, stretching, or other discontinuities of an infusion sleeve that is formed in a first shape, and then deflected into a use shape, e.g., a curved position, will generally exhibit variations in material stress and/or strain along the deflected regions.

In another exemplary illustration, the tubular body 101 is formed in a curved position such that the first axis A-A and the second axis B-B define the predetermined angle α when the tubular body 101 is not constrained or deflected out of a natural position associated with the tubular body 101. For example, prior to assembly of the tubular body 101 to the aspiration tube 114, the natural tendency of the tubular body 101, e.g., due to the resiliency of the material and the curved shape in which the tubular body 101 is initially formed, is to assume the curved position generally shown in the figures. A resilient property of the material forming the tubular body 101 may thus cause the tubular body 101 to maintain the curved position when the tubular body 101 is deflected out of the curved position and then released. In other words, to the extent that the tubular body 101 must be flexible to generally permit working of the material as it is moved over the aspiration tube 114 and into position, the material may be generally resilient so that it assumes the curved position in which the tubular body is formed. As shown in FIGS. 2 and 3A, the curve of the tubular body 101 and/or curved portion 110 may generally mimic the curve of the aspiration tube 114.

As briefly described above, the infusion sleeve assembly 100 may generally be used during cataract surgery to aspirate and irrigate a surgical site. In one exemplary illustration, the infusion sleeve assembly 100 is inserted into an incision formed on or around an eye such that the tip and a portion of the tubular body 101 are inserted into an associated ocular cavity. Irrigation fluid may be delivered to the site via the irrigation path P_{I}, with the irrigation fluid exiting the infusion sleeve assembly 100 at the fluid aperture 108. Fluid may further be aspirated from the site via the aspiration path P_{A}, being drawn into the fluid aperture 116 of the tip 112 and back into the surgical tool 200 via the aspiration sleeve 114.

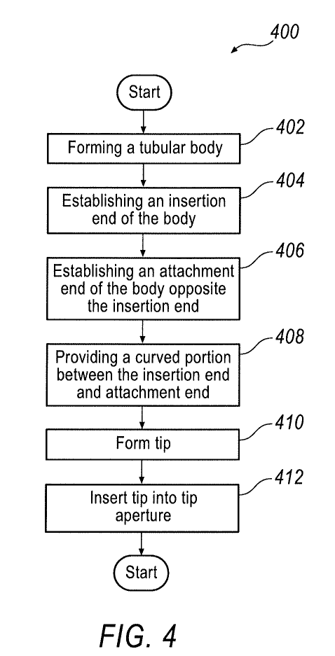

Turning now to FIG. 4, an exemplary process 400 for assembling an infusion sleeve assembly will be described. Process 400 may begin at block 402, where a tubular body 101 is formed, e.g. of a single flexible and/or resilient material. In examples where the tubular body 101 is formed of a single material, the single material may define a substantially constant material strain throughout the curved portion. For example, as described above, the curved portion 110 may be formed of a single flexible and/or resilient material that defines a substantially constant material strain throughout the curved portion 110. Accordingly, the curved portion 110 does not exhibit bunching, stretching, or other distortions of the material, especially in the outer surface S that would otherwise interfere with insertion/removal of the infusion sleeve assembly 100 to/from an incision, respectively.

Additionally, in one exemplary illustration a tubular body 101 formed of a single material may be substantially resilient. For example, the tubular body 101 may be formed of a silicone, rubber or other resilient material such that the tubular body 101 tends to assume the curved position when the tubular body 101 is deflected out of the curved position and subsequently released or unconstrained. Process 400 may then proceed to block 404.

In block 404, the body 101 may be established with an insertion end 102 having a tip aperture 106. The insertion end 102 may further be configured to include a first fluid aperture 108 that is adjacent the tip aperture 106. Process 400 may then proceed to block 406.

At block 406, an attachment end 104 of the tubular body 101 may be established. For example, as described above, the tubular body 101 may be formed with an attachment end 104 generally opposite the insertion end 102. The attachment end 104 may be configured to be secured to a surgical tool 200 for fluid exchange with the surgical tool 200, e.g., as in a cataract surgery procedure as described above. Process 400 may then proceed to block 408.

At block 408, a curved portion 110 is provided between the insertion end 102 and the attachment 104. For example, as described above, tubular body 101 may be formed with a curved portion 110 such that a first axis A-A of the insertion end 102 is angled relative to a second axis B-B of the attachment end 104. The curved portion 110 may be formed of a single material that generally defines a substantially smooth body surface S throughout the curved portion 110. For example, as described above, the tubular body 101 may be formed in a curved position such that the first axis A-A and the second axis B-B define the predetermined angle α when the tubular body 101 is not constrained or deflected. For example, where the tubular body is formed in a molding operation, the mold may generally define the curved position shown in the Figures. Any suitable operation may be employed to form the curved portion 110. Process 400 may then proceed to block 410.

In block 410, a tip may be formed, e.g., tip 112 as described above. For example, a tip 112 may be formed of a plastic or nylon material. The tip 112 may define a second fluid aperture 116, e.g., that is radially spaced away from the first fluid aperture 108 of the tubular body 101. The tip 112 may be configured to receive an aspiration tube, e.g. aspiration tube 114, which extends within the infusion sleeve 101. In one exemplary illustration, the tip 112 may have a flared shoulder portion 118 adjacent the insertion end 102. The flared shoulder portion 118 may be angled to provide an angled surface extending between a relatively narrow diameter associated with the tip 112 and a second larger diameter of the tubular body 101, e.g., diameter D2, thereby providing a substantially smooth interface or transition between the tip 112 and tubular body 101.

The tip 112 may have a tip outer diameter, e.g. diameter D₁ that is substantially equal to an outer diameter of the insertion end 102, e.g. outer diameter D₂. For example, an outer diameter D₁ of tip 112 measured at an end of the tip 112 adjacent tubular body 101 may be substantially equal to the outer diameter D₂ of the insertion end 102, such that the tip 112 and the tubular body 101 define a substantially smooth surface between the tip 112 and tubular body 101. Process 400 may then proceed to block 412.

In block 412, the tip 112 may be inserted into the tip aperture 106. The tip 112 may include a male connector 120 that is received within the tip aperture 106.

The male connector 120 may have an outer diameter substantially equal to an inner diameter of the insertion end 102 of the tubular body 101, as described above. Process 400 may then terminate.

Accordingly, an exemplary infusion sleeve assembly 100 and corresponding process 400 may allow for a curved infusion sleeve that minimizes or eliminates distortion along an outer surface S of the infusion sleeve assembly 100. The smooth outer surface S thereby minimizes distortion or irritation to incisions created during cataract surgery.

Reference in the specification to “one example,” “an example,” “one embodiment,” or “an embodiment” means that a particular feature, structure, or characteristic described in connection with the example is included in at least one example. The phrase “in one example” in various places in the specification does not necessarily refer to the same example each time it appears.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claimed invention.

Accordingly, it is to be understood that the above description is intended to be illustrative and not restrictive. Many embodiments and applications other than the examples provided would be upon reading the above description. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future embodiments. In sum, it should be understood that the invention is capable of modification and variation and is limited only by the following claims.

All terms used in the claims are intended to be given their broadest reasonable constructions and their ordinary meanings as understood by those skilled in the art unless an explicit indication to the contrary in made herein. In particular, use of the singular articles such as “a,” “the,” “said,” etc. should be read to recite one or more of the indicated elements unless a claim recites an explicit limitation to the contrary.

What is claimed is:

1. An infusion sleeve, comprising:
a tubular body, the body including:
an insertion end defining a tip aperture, the insertion end defining a first fluid aperture adjacent the tip aperture;
an attachment end opposite the insertion end, the attachment end configured to be secured to a surgical tool for fluid exchange therewith; and
a curved portion between the insertion end and attachment end such that a first axis of the insertion end defines a predetermined angle relative to a second axis of the attachment end;
wherein the tubular body defines a substantially smooth body surface throughout the curved portion.

2. The infusion sleeve of claim 1, further comprising a tip inserted into the tip aperture and configured to receive an aspiration tube extending within the infusion sleeve, the tip defining a second fluid aperture.

3. The infusion sleeve of claim 2, wherein the tip defines a flared shoulder portion adjacent the insertion end.

4. The infusion sleeve of claim 3, wherein the flared shoulder portion has an outer diameter substantially equal to an outer diameter of the insertion end, thereby defining a substantially smooth surface at an interface between the tip and the tubular body.

5. The infusion sleeve of claim 2, wherein the tip includes a male connector inserted into the tip aperture.

6. The infusion sleeve of claim 5, wherein the male connector defines an outer diameter substantially equal to an inner diameter of the insertion end of the tubular body.

7. The infusion sleeve of claim 1, wherein the tubular body includes a single material and defines a substantially constant strain in the single material throughout the curved portion.

8. The infusion sleeve of claim 7, wherein the single material is substantially resilient such that the tubular body tends to the curved position when the tubular body is deflected out of the curved position.

9. The infusion sleeve of claim 1, wherein the tubular body is formed in a curved position such that the first axis and the second axis define the predetermined angle when the tubular body is not constrained.

10. The infusion sleeve of claim 1, wherein the tubular body includes a silicone material.

11. A method, comprising:
forming a tubular body, including:
establishing an insertion end of the body with a tip aperture, the insertion end defining a first fluid aperture adjacent the tip aperture;
establishing an attachment end of the body opposite the insertion end, the attachment end configured to be secured to a surgical tool for fluid exchange therewith; and
providing a curved portion between the insertion end and attachment end such that a first axis of the insertion end is angled relative to a second axis of the attachment end, the tubular body defining a substantially smooth body surface throughout the curved portion.

12. The method of claim 11, further comprising inserting a tip into the tip aperture, the tip configured to receive an aspiration tube extending within the infusion sleeve, the tip defining a second fluid aperture.

13. The method of claim 12, wherein the tip includes a flared shoulder portion adjacent the insertion end.

14. The method of claim 12, wherein the tip includes a tip outer diameter substantially equal to a body outer diameter of the insertion end, the tip outer diameter measured adjacent an interface between the tip and the tubular body such that the tip and tubular body define a substantially smooth surface across the interface.

15. The method of claim 12, wherein inserting the tip into the tip aperture includes inserting a male connector of the tip into the tip aperture.

16. The method of claim 15, wherein the male connector includes an outer diameter substantially equal to an inner diameter of the insertion end of the tubular body.

17. The method of claim 11, wherein forming the tubular body includes forming the tubular body from a single material, the single material defining a substantially constant material strain throughout the curved portion.

18. The method of claim 17, further comprising establishing the single material as substantially resilient such that the tubular body tends to the curved position when the tubular body is deflected out of the curved position.

19. The method of claim 11, wherein forming the tubular body includes forming the tubular body in a curved position such that the first axis and the second axis define the predetermined angle when the tubular body is not constrained.

20. An infusion sleeve assembly, comprising:
a tubular body, including:
an insertion end configured to be inserted into an operating site, the insertion end defining a tip aperture, the insertion end defining a first fluid aperture adjacent the tip aperture;
an attachment end opposite the insertion end, the attachment end configured to be secured to a surgical tool for fluid exchange therewith; and
a curved portion between the insertion end and attachment end such that a first axis of the insertion end is angled relative to a second axis of the attachment end; and
a tip inserted into the tip aperture and configured to receive an aspiration tube extending within the infusion sleeve, the tip defining a second fluid aperture and having a flared shoulder portion adjacent the insertion end, the flared shoulder portion having an outer diameter substantially equal to an outer diameter of the insertion end;
wherein the tubular body and tip cooperate to define a substantially smooth surface throughout the curved portion and an interface between the tip and the body.
